(19) United States
(12) Patent Application Publication
(10) Pub. No.: US 2022/0289682 A1
(43) Pub. Date: Sep. 15, 2022

(54) MOLECULAR COMPLEXES

(71) Applicant: Johnson Matthey Public Limited Company, London (GB)

(72) Inventors: Thierry BONNAUD, Cambridge (GB); Alan CHORLTON, Cambridge (GB); Thomas KENDALL, Cambridge (GB); Jose VILLORIA, Reading (GB)

(21) Appl. No.: 17/632,580

(22) PCT Filed: Aug. 6, 2020
(86) PCT No.: PCT/GB2020/051877

§ 371 Date: Feb. 3, 2022

(30) Foreign Application Priority Data
Aug. 7, 2019 (GB) 1911300.0

(51) Int. Cl. C07D 231/12; A01N 43/56

(52) U.S. Cl. CPC C07D 231/12; A01N 43/56; C07B 2200/13

(57) ABSTRACT

The present invention relates to a crystalline molecular complex comprising metazachlor and a carboxylic acid. The invention also relates to a process for the preparation of the crystalline molecular complex, a herbicidal composition comprising the crystalline molecular complex, and use of the herbicidal composition.

The present invention relates to molecular complexes of the herbicide metazachlor. In particular, the invention relates to molecular complexes comprising metazachlor with carboxylic acids, and to herbicidal compositions comprising such molecular complexes.

Metazachlor has the chemical name 2-chloro-N-(2,6-dimethylphenyl)-N-(pyrazol-1-ylmethyl)acetamide and the chemical structure illustrated below:

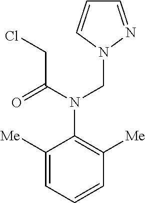 [SMILES: Cc1cccc(C)c1N(Cn1cccn1)C(=O)CCl]

U.S. Pat. No. 4321395 (to BASF Aktiengesellschaft) describes a method for the preparation of acetanilides, including metazachlor, by reacting 2-halo-N-halomethylacetanilides with azoles in a two-phase system, in the presence or absence of a phase transfer catalyst.

### Definitions

The term “about” or “approximately” means an acceptable error for a particular value as determined by a person of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range. In certain embodiments and with reference to X-ray powder diffraction two-theta peaks, the terms “about” or “approximately” means within ±0.2° 2θ.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like.

The term “ambient temperature” means one or more room temperatures between about 15° C. to about 30° C., such as about 15° C. to about 25° C.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Arylalkyl” refers to an optionally substituted group of the formula aryl-alkyl-, where aryl and alkyl are as defined above.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

“Halo”, “hal” or “halide” refers to —F, —Cl, —Br and —I.

The term “molecular complex” is used to denote a crystalline material composed of two or more different components which has a defined single-phase crystal structure. The components are held together by non-covalent bonding, such as hydrogen bonding, ionic bonding, van der Waals interactions, Tr-Tr interactions, etc. The term “molecular complex” includes salts, co-crystals and salt/co-crystal hybrids. In one embodiment, the molecular complex is a salt. In another embodiment, the molecular complex is a co-crystal. In another embodiment, the molecular complex is a salt/co-crystal hybrid.

Without wishing to be bound by theory, it is believed that when the molecular complex is a co-crystal, the co-crystal demonstrates improved physiochemical properties, such as crystallinity, solubility properties and/or modified melting points. In certain embodiments, the melting point of the molecular complex may be higher than the melting point of metazachlor itself. In this instance, a higher melting point may be of benefit in the preparation of, for example, a suspension concentrate formulation of the molecular complex. In certain embodiments, the melting point of the molecular complex may be lower than the melting point of metazachlor itself. In this instance, a lower melting point may be of benefit in the preparation of, for example, an encapsulated formulation of the molecular complex or liquid formulation of the molecular complex.

The molecular complexes may be distinguished from mixtures of metazachlor and the selected molecular complex former, such as a carboxylic acid, by standard analytical means which are well known to those skilled in the art, for example X-ray powder diffraction (XRPD), single crystal X-ray diffraction, or differential scanning calorimetry (DSC). The molar ratio of the components of the molecular complex may be determined using, for example, HPLC or ¹H-NMR.

The term “overnight” refers to the period of time between the end of one working day to the subsequent working day in which a time frame of about 12 to about 18 hours has elapsed between the end of one procedural step and the instigation of the following step in a procedure.

“Slurry” means a heterogeneous mixture of at least a portion of the molecular complex in one or more solvents. “Slurry” therefore includes a mixture of molecular complex which is partially present as a solid, as well as being partially dissolved in the one or more solvents.

“Substituted” refers to a group in which one or more hydrogen atoms are each independently replaced with substituents (e.g. 1, 2, 3, 4, 5 or more) which may be the same or different. The group may be substituted with one or more substituents up to the limitations imposed by stability and the rules of valence. The substituents are selected such that they do not adversely affect the molecular complexes.

Examples of substituents include but are not limited to -halo, —CF₃, —R^{a}, —O——R^{a}, -S—R^{a}, —NR^{a}R^{b}, —CN, —C(O)—R^{a}, —COOR^{a}, and —CONR^{a}R^{b}, preferably -halo, —CF₃, —R^{a}, —O—R^{a}, —NR^{a}R^{b}, —COOR^{a}, and —CONR^{a}R^{b}. R^{a} and R^{b} are independently selected from the groups consisting of H, alkyl, aryl, and arylalkyl, and wherein R^{a} and R^{b} may be unsubstituted or further substituted as defined herein.

### DESCRIPTION OF THE FIGURES

FIG. 1 shows a representative x-ray diffraction pattern (XRPD) of the molecular complex of Example 1.

FIG. 2 shows a representative differential scanning calorimetry (DSC) curve for the molecular complex of Example 1.

FIG. 3 shows a representative x-ray diffraction pattern (XRPD) of the molecular complex of Example 2.

FIG. 4 shows a representative differential scanning calorimetry (DSC) curve for the molecular complex of Example 2.

FIG. 5 shows a representative x-ray diffraction pattern (XRPD) of the molecular complex of Example 3.

FIG. 6 shows a representative differential scanning calorimetry (DSC) curve for the molecular complex of Example 3.

FIG. 7 shows a representative x-ray diffraction pattern (XRPD) of the molecular complex of Example 4.

FIG. 8 shows a representative differential scanning calorimetry (DSC) curve for the molecular complex of Example 4.

FIG. 9 shows a representative x-ray diffraction pattern (XRPD) of the molecular complex of Example 5.

FIG. 10 shows a representative differential scanning calorimetry (DSC) curve for the molecular complex of Example 5.

FIG. 11 shows a view of the asymmetric unit of the crystal structure for metazachlor:benzoic acid molecular complex of Example 1.

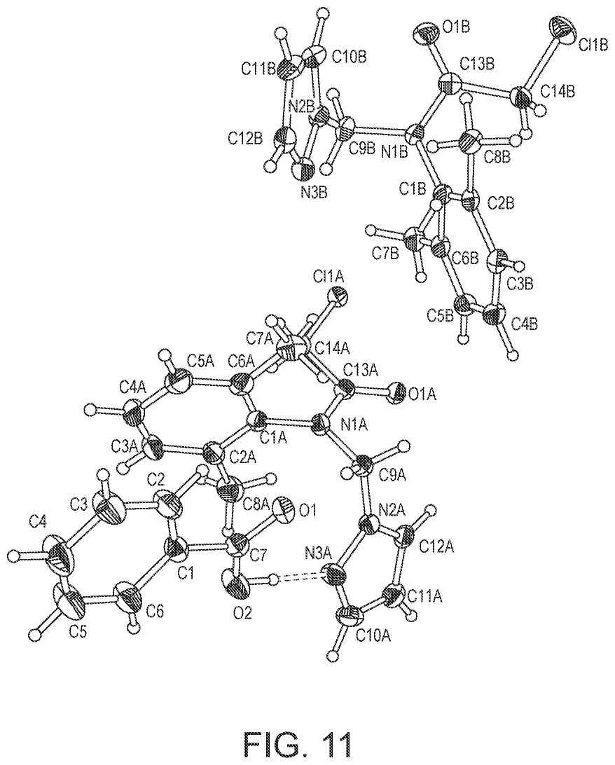

FIG. 12 shows a view of the asymmetric unit of the crystal structure for metazachlor:fumaric acid molecular complex of Example 2.

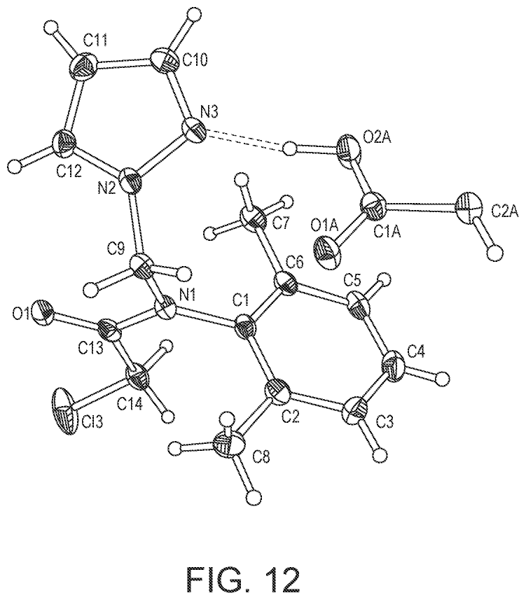

### DESCRIPTION OF THE INVENTION

It is an object of the present invention to provide a crystalline molecular complex of the herbicide metazachlor and a carboxylic acid. In certain embodiments, the crystalline molecular complex is purifiable. In certain embodiments, the crystalline molecular complex facilitates obtaining metazachlor in an improved purity. In certain embodiments, the crystalline molecular complex is stable. In certain embodiments, the crystalline molecular complex is easy to isolate and handle. In certain embodiments, the process for preparing the crystalline molecular complex is scalable. In certain embodiments, the dissolution rates of the crystalline molecular complex is higher than the dissolution rate of metazachlor itself.

The crystalline forms described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The purity of the crystalline forms provided herein may be determined by standard analytical methods, such as thin layer chromatography (TLC), gas chromatography, high performance liquid chromatography (HPLC), and mass spectrometry (MS).

In one aspect, the present invention provides a crystalline molecular complex comprising metazachlor and a carboxylic acid.

A soil pollutant is a persistent toxic material which causes deterioration or loss of one or more soil functions above a certain level. The carboxylic acid in the molecular complex of the present invention does not become a soil pollutant, or does not degrade into a soil pollutant, after the administration of a herbicidally effective amount of the molecular complex to undesired vegetation growing in soil, compost, or other plant-growing medium.

The molar ratio of the metazachlor:carboxylic acid may be from about 0.1:about 5 to about 5:about 0.1. In one embodiment, the molar ratio of the metazachlor:carboxylic acid may be about 2:about 1.

The carboxylic acid may be selected from the group consisting of:
(a) the compound of formula (2):

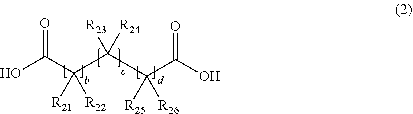

wherein:
b is an integer which is 0, 1, 2, 3, 4, or 5;
c is an integer which is 0, 1, 2, 3, 4, or 5;
d is an integer which is 0, 1, 2, 3, 4, or 5;
R₂₁, R₂₂, R₂₃, R₂₄, R₂₅, and R₂₆ are independently selected from the group consisting of —H, —OH, —NH₂ and —COOH; or
the pair of R₂₁/R₂₂, R₂₃/R₂₄ and/or R₂₅/R₂₆ independently is a carbonyl

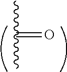

group.
(b) the compound of formula (3):

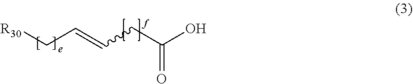

wherein:
e is an integer selected from 0, 1, 2, 3, 4, 5, 6, 7, or 8;
f is an integer selected from 0, 1, 2, 3, 4, 5, 6, 7, or 8;
the symbol [symbol] denotes that the stereochemistry of the C═C double bond is cis- or trans-;
R₃₀ is selected from the group consisting of —H, —CO₂H, unsubstituted C₁-C₂₀-alkyl, substituted C₁-C₂₀-alkyl, unsubstituted C₅-C₂₀-aryl, and substituted C₅-C₂₀-aryl.
(c) the compound of formula (4):

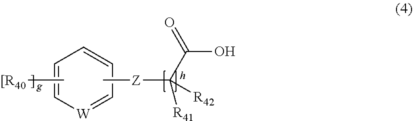

wherein:
g is an integer selected from 0, 1, 2, 3, 4 or 5;
h is an integer selected from 0, 1, 2, 3, or 4;
W is a carbon atom or a nitrogen atom;
Z is absent or is a —CO—NH— group;
R₄₀ is selected from the group consisting of —OH, —NH₂, and —NH—CO—R₄₃;
R₄₁ and R₄₂ are independently selected from the group consisting of —H, —OH, unsubstituted C₁-C₂₀-alkyl, substituted C₁-C₂₀-alkyl, unsubstituted C₅-C₂₀-aryl, and substituted C₅-C₂₀-aryl;
R₄₃ is selected from the group consisting of unsubstituted C₁-C₂₀-alkyl, substituted C₁-C₂₀-alkyl, unsubstituted C₅-C₂₀-aryl, and substituted C₅-C₂₀-aryl;
provided that when W is a nitrogen atom, g is an integer selected from 0, 1, 2, 3, or 4.
(d) the compound of formula (5):

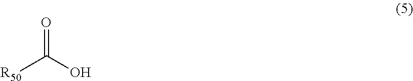

wherein:
R₅₀ is selected from the group consisting of —H, unsubstituted C₁-C₂₀-alkyl, and substituted C₁-C₂₀-alkyl.
(e) the compound of formula (6):

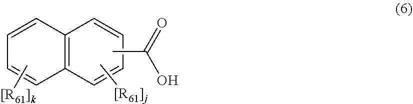

wherein:
j is an integer selected from 0, 1, 2 or 3;
k is an integer selected from 0, 1, 2, 3, or 4;
R₆₀ and R₆₁ are independently selected from the group consisting of —H, unsubstituted C₁-C₂₀-alkyl, substituted C₁-C₂₀-alkyl, unsubstituted C₅-C₂₀-aryl, substituted C₅-C₂₀-aryl, unsubstituted —(C₁-C₂₀-alkyl)-(C₅-C₂₀-aryl), and substituted —(C₁-C₂₀-alkyl)-(C₅-C₂₀-aryl).
(f) the compound of formula (7):

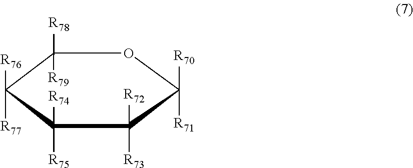

wherein:
R_{79,} R₇₁, R₇₂, R₇₃, R₇₄, R₇₅, R₇₆, R₇₇, R₇₈ and R₇₉ are independently selected from the group consisting of —H, —OH and —COOH,
provided that at least one of R₇₀, R₇₁, R₇₂, R₇₃, R₇₄, R₇₅, R₇₆, R₇₇, R₇₈ and R₇₉ is —COOH.
(g) the compound of formula (8):

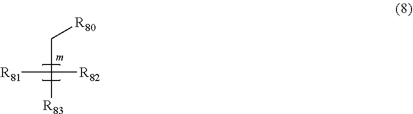

wherein:
m is an integer selected from 1, 2, 3, 4, 5, 6, 7, 8, 9, or 10;
R₈₀, R₈₁, R₈₂ and R₈₃ are independently selected from —H, —OH, —COOH and R_{84;}
R₈₄ is the group:

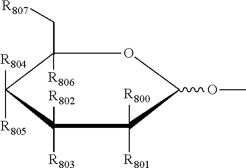

wherein:
R₈₀₀, R₈₀₁, R₈₀₂, R₈₀₃, R₈₀₄, R₈₀₅, R₈₀₆ and R₈₀₇ are independently selected from the group consisting of —H, —OH, and —COOH;
provided that at least one of R₈₀, R₈₁, R₈₂, R₈₃, R₈₀₀, R₈₀₁, R₈₀₂, R₈₀₃, R₈₀₄, R₈₀₅, R₈₀₆ and R₈₀₇ is —COOH.
(h) the compound of formula (9):

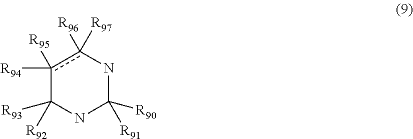

wherein:
[symbol] is a single or double bond;
R₉₀, R₉₁, R₉₂, R₉₃, R₉₄, R₉₅, R₉₆, and R₉₇ are independently selected from the group consisting of —H, unsubstituted C₁-C₂₀-alkyl, substituted C₁-C₂₀-alkyl, and —COOH; or
the pair of R₉₀/R₉₁, R₉₂/R₉₃, R₉₄/R₉₅ and/or R₉₆/R₉₇ independently is a carbonyl

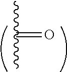

group;
provided that:
(i) when [symbol] is a double bond, R₉₅ and R₉₇ are absent;
(ii) when the pair of R₉₄/R₉₅ and/or R₉₆/R₉₇ is a carbonyl

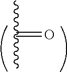

group, [symbol] is a single bond;
(iii) at least one of R₉₀, R₉₁, R₉₂, R₉₃, R₉₄, R₉₅, R₉₆, and R₉₇ is —COOH.
(g) the compound of formula (10):

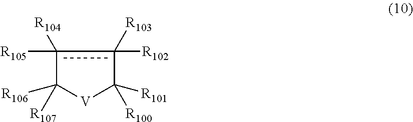

wherein:
[symbol] is a single or double bond;
V is —O—,

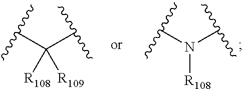

R₁₀₀, R₁₀₁, R₁₀₂, R₁₀₃, R₁₀₄, R₁₀₅, R₁₀₆, R₁₀₇, Rios, Rio9 and R₁₁₀ are independently selected from the groups consisting of —H, —OH, unsubstituted C₁-C₂₀-alkyl, substituted C₁-C₂₀-alkyl, and —COOH; or
the pair of R₁₀₀/R₁₀₁, R₁₀₂/R₁₀₃, R₁₀₄/R₁₀₅ or R₁₀₆/R₁₀₇ independently is a carbonyl

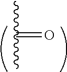

group;
provided that:
(i) when [symbol] is a double bond, R₁₀₂ and R₁₀₅ are absent; and
(ii) when the pair of R₁₀₄/R₁₀₅ and/or R₁₀₆/R₁₀₇ is a carbonyl

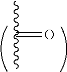

group, [symbol] is a single bond.

### Compound of Formula (2)

When b is selected from 2, 3, 4, or 5, each R₂₁ may be the same or different to every other R₂₁, and each R₂₂ may be the same or different from every other R₂₂. When c is selected from 2, 3, 4, or 5, each R₂₃ may be the same or different to every other R₂₃, and each R₂₄ may be the same or different from every other R₂₄. When d is selected from 2, 3, 4, or 5, each R₂₅ may be the same or different to every other R₂₅, and each R₂₆ may be the same or different from every other R_{26.}

When one of R₂₁ is different from R₂₂, and/or R₂₃ is different from R₂₄, and/or R₂₅ is different from R₂₆, the compound (2) will have one or more chiral carbon atoms. Racemates, enantiomers and diastereomers are within the scope of the invention.

In one embodiment, the compound of formula (2) may be a compound of formula (2a):

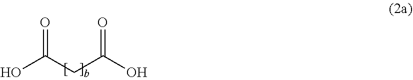

wherein:

b is an integer which is 0, 1, 2, 3, 4, or 5.

When b is 0, the compound of formula (2a) is oxalic acid i.e. the —COOH groups are bonded directly to each other. When b is 1, the compound of formula (2a) is malonic acid. When b is 2, the compound of formula (2a) is succinic acid. When b is 3, the compound of formula (2a) is glutaric acid. When b is 4, the compound of formula (3a) is adipic acid. When b is 5, the compound of formula (2a) is pimelic acid.

In another embodiment, the compound of formula (2) may be a compound of formula (2b):

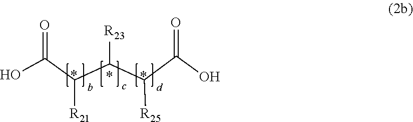

wherein:

* denotes a chiral carbon atom, which may have the same or different stereochemistry to any other chiral carbon atom present in the compound;

b is an integer which is 0, 1, 2, 3, 4, or 5;

c is an integer which is 0, 1, 2, 3, 4, or 5;

d is an integer which is 0, 1, 2, 3, 4, or 5;

R₂₁, R₂₃, and R₂₅ are independently selected from the group consisting of —OH, —NH₂ and —COOH.

When b and/or d are not 0 (i.e. are independently selected from 1, 2, 3, 4, or 5), R₂₂, R₂₄ and R₂₆ are all —H (and, as such, the hydrogen atoms are not specifically shown in formula (2b)).

Examples of compound of formula (2b) include but are not limited to DL-aspartic acid, L-aspartic acid, D-aspartic acid, DL-glutamic acid, L-glutamic acid, D-glutamic acid, DL-tartaric acid, D-(−)-tartaric acid, L-(+)-tartaric acid, galactaric acid (also known as mucic acid), DL-malic acid, D-malic acid, or L-malic acid.

In another embodiment, the compound of formula (2) may be a compound of formula (2c):

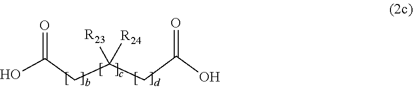

wherein:

b is an integer which is 0, 1, 2, 3, 4, or 5;

c is an integer which is 0, 1, 2, 3, 4, or 5;

d is an integer which is 0, 1, 2, 3, 4, or 5;

R₂₃ and R₂₄ are independently selected from the group consisting of —H, —OH, —NH₂ and —COOH;

provided that when one of R₂₃ and R₂₄ is —H, the other of R₂₃ and R₂₄ is selected from the group consisting of —OH, —NH₂ and —COOH; or

the pair of R₂₃/R₂₄ is a carbonyl

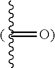

group.

When b and/or d are not 0 (i.e. are independently selected from 1, 2, 3, 4, or 5), R₂₁, R₂₂, R₂₅ and R₂₆ are —H (and, as such, the hydrogen atoms are not specifically shown in formula (2c)).

Examples of compound of formula (2c) include but are not limited to ketoglutaric acid, or citric acid.

### Compound of Formula (3)

In one embodiment, R₃₀ is selected from the group consisting of —H, —CO₂H, unsubstituted C₁-C₂₀-alkyl, and unsubstituted C₅-C₂₀-aryl. In another embodiment, R₃₀ is selected from the group consisting of —H, —CO₂H and —Me.

In one embodiment, the compound of formula (3) has a cis-stereochemistry i.e.

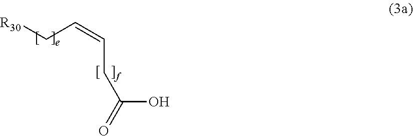

In this instance, R₃₀ may be selected from the group consisting of —COOH, unsubstituted C₁-C₂₀-alkyl, and unsubstituted C₅-C₂₀-aryl, for example, —COOH, —Me or —Ph.

Examples of compound of formula (3a) include but are not limited to maleic acid, oleic acid, or cis-cinnamic acid.

In one embodiment, the compound of formula (3) has a trans- stereochemistry i.e.

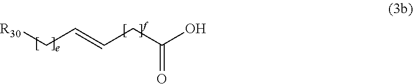

In this instance, R₃₀ may be selected from the group consisting of —COOH, unsubstituted C₁-C₂₀-alkyl, and unsubstituted C₅-C₂₀-aryl, for example, —COOH, —Me or —Ph.

Examples of compound of formula (3b) include but are not limited to fumaric acid, or trans-cinnamic acid.

In one embodiment, e is 0 and R₃₀ is —H. In one instance, the compound (3) may be undecylenic acid.

### Compound of Formula (4)

In one embodiment, the compound of formula (4) may be a compound of formula (4a):

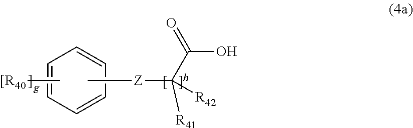

wherein:

g is an integer selected from 0, 1, 2, 3, 4 or 5;

h is an integer selected from 0, 1, 2, 3, or 4;

R₄₀ is selected from the group consisting of —OH, —NH₂, and —NH—CO—R_{43;}

R₄₁ and R₄₂ are independently selected from the group consisting of —H, —OH, unsubstituted C₁-C₂₀-alkyl, substituted C₁-C₂₀-alkyl, unsubstituted C₅-C₂₀-aryl, and substituted C₅-C₂₀-aryl; and R₄₃ is selected from the group consisting of unsubstituted C₁-C₂₀-alkyl, substituted C₁-C₂₀-alkyl, unsubstituted C₅-C₂₀-aryl, and substituted C₅-C₂₀-aryl.

In this instance, W is a carbon atom i.e. the 6-membered ring containing W is an aryl group.

In one embodiment, Z is absent (i.e. the

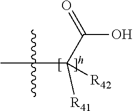

group is bonded directly to the aryl ring). In another embodiment, Z is a —CO—NH— group.

In one embodiment, R₄₀ may be selected from the group consisting of —OH, —NH₂ and —NH—CO—Me.

R₄₀ may be present or absent. When absent, g is 0 i.e. the aromatic ring is unsubstituted by R₄₀ groups. When R₄₀ is present, g may be 1, 2, 3, 4, or 5. In one embodiment, the g is an integer selected from 0, 1, or 2.

When h is 0, the —COOH group may be bonded to —Z— or, if Z is absent, directly to the aromatic ring. In one embodiment, h is selected from 0 or 1. In one embodiment, h is 0 and g is selected from 0, 1 or 2. In one embodiment, h is 1 and g is selected from 0, 1, or 2.

When h is selected from 2, 3, or 4, each R₄₁ may be the same or different to every other R₄₁, and each R₄₂ may be the same or different from every other R₄₂. When one of R₄₁ is different from R₄₂, the compound (4a) will have one or more chiral carbon atoms. Racemates, enantiomers and diastereomers are within the scope of the invention.

In one embodiment, R₄₁ and R₄₂ are independently selected from the group consisting of —H, and —OH. In another embodiment, one of R₄₁ and R₄₂ is —H and the other of R₄₁ and R₄₂ is —OH.

In one embodiment, R₄₁ and R₄₂ are both —H.

Examples of compound of formula (4a) include but are not limited to benzoic acid, dihydroxybenzoic acid (e.g. 2,3-, 2,4-, 2,5-, 2,6-, 3,4- or 3,5-dihydroxybenzoic acid), hydroxybenzoic acid (e.g. 2-, 3- or 4-hydroxybenzoic acid), amino-hydroxybenzoic acid (e.g. 4-amino-2-hydroxybenzoic acid), mandelic acid (e.g. D-, L- or DL-mandelic acid), acetamidobenzoic acid (e.g. 2-, 3- or 4-acetamidobenzoic acid), and hippuric acid.

In one embodiment, the compound of formula (4) may be a compound of formula (4b):

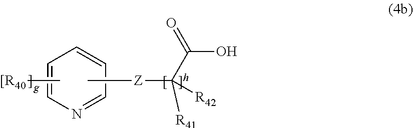

wherein:

g is an integer selected from 0, 1, 2, 3, or 4;

h is an integer selected from 0, 1, 2, 3, or 4;

Z is absent or is a —CO—NH— group;

R₄₀ is selected from the group consisting of —OH, —NH₂, and —NH—CO—R_{43;}

R₄₁ and R₄₂ are independently selected from the group consisting of —H, —OH, unsubstituted C₁-C₂₀-alkyl, substituted v-alkyl, unsubstituted C₅-C₂₀-aryl, and substituted C₅-C₂₀-aryl; and

R₄₃ is selected from the group consisting of unsubstituted C₁-C₂₀-alkyl, substituted C₁-C₂₀-alkyl, unsubstituted C₅-C₂₀-aryl, and substituted C₅-C₂₀-aryl.

In this instance, W is a nitrogen atom i.e. the 6-membered ring containing W is a pyridinyl ring.

g, h, Z R₄₀, R₄₁, R₄₂ and R₄₃ are as described above for the compound of formula (4a).

In one embodiment, the compound of formula (4b) may be nicotinic acid.

### Compound of Formula (5)

In one embodiment, Fin is selected from the group consisting of —H, unsubstituted C₁-C₂₀-alkyl, and substituted C₁-C₂₀-alkyl, and the substituents are selected from the group consisting of —OH and —NH₂.

Examples of compound of formula (5) include but are not limited to formic acid, glycolic acid, acetic acid, caproic acid, propionic acid, stearic acid or caprylic acid.

### Compound of Formula (6)

R₆₀ may be present or absent. When absent, j is 0 i.e. the aromatic ring is unsubstituted. When R₆₀ is present, j may be 1, 2, or 3. In one embodiment, the j is an integer selected from 0 or 1.

R₆₀ may be selected from the group consisting of —OH and substituted —(C₁-C₂₀-alkyl)-(C₅-C₂₉-aryl), wherein the substituents are selected from the group consisting of —OH and —COOH. An example of a substituted —(C₁-C₂₉-alkyl)-(C₅-C₂₉-aryl) group includes but is not limited to -CH2-(3-hydroxy-2-naphthoic acid).

R₆₁ may be present or absent. When absent, k is 0 i.e. the aromatic ring is unsubstituted. When R₆₁ is present, k may be 1, 2, 3, or 4.

R₆₁ may be selected from the group consisting of —OH and substituted —(C₁-C₂₉-alkyl)-(C₅-C₂₉-aryl), wherein the substituents are selected from the group consisting of —OH and —COOH. An example of a substituted —(C₁-C₂₉-alkyl)-(C₅-C₂₉-aryl) group includes but is not limited to —CH₂-(3-hydroxy-2-naphthoic acid).

In one embodiment, the compound of formula (6) may be a compound (6a):

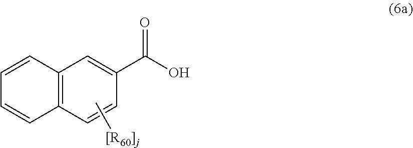

R₆₀ and j are as described above. In this instance, R₆₁ is absent i.e. k is 0.

Examples of compounds (6) include but are not limited to hydroxy-naphthoic acid (e.g. 1-hydroxy-2-napthoic acid) and pamoic acid.

### Compound of Formula (7)

One of R₇₉ and R₇₁ may be —H and the other of R₇₀ and R₇₁ may be —OH. One of R₇₂ and R₇₃ may be —H and the other of R₇₂ and R₇₃ may be —OH. One of R₇₄ and R₇₅ may be —H and the other of R₇₄ and R₇₅ may be —OH. One of R₇₆ and R₇₇ may be —H and the other of R₇₆ and R₇₇ may be —OH. One of R₇₈ and R₇₉ may be —H and the other of R₇₈ and R₇₉ may be —COOH.

An example of compound of formula (7) includes but is not limited to D-glucuronic acid.

### Compound of Formula (8)

When m is 2, 3, 4, 5, 6, 7, 8, 9, or 10, each R₈₁ may be the same or different to every other R₈₁, and each R₈₂ may be the same or different from every other R₈₂. When one of R₈₁ is different from R₈₂, the compound (8) will have one or more chiral carbon atoms. Racemates, enantiomers and diastereomers are within the scope of the invention.

Compound (8) may be substituted with one or more R₈₄ groups up to the limitations imposed by stability, for example, one R₈₄ group or more than one (e.g. two R₈₄ groups).

The glycosidic bond ( [symbol] ) at the animeric carbon atom of R₈₄ group may be in the α- or β-position.

One of R₈₀₀ and RR₈₀₁ may be —H and the other of R₈₀₀ and R₈₀₁ may be —OH. One of R₈₀₂ and R₈₀₃ may be —H and the other of R₈₀₂ and R₈₀₃ may be —OH. One of R₈₀₁ and R₈₀₅ may be —H and the other of R₈₀₄ and R₈₀₅ may be —OH. R₈₀₆ and R₈₀₇ may be selected from —H or —OH. In one embodiment, R₈₀₆ is —H and R₈₀₇ is —OH.

Examples of compound of formula (8) include but are not limited to lactic acid (e.g. DL-, D- or L-lactic acid), glucoheptonic acid (e.g. D-glucoheptonic acid), lactobionic acid, and gluconic acid (e.g. DL-, D- or L-gluconic acid).

### Compound of Formula (9)

In one embodiment, R₉₀, R₉₁, R₉₂, R₉₃, R₉₄, R₉₅, R₉₆, and R₉₇ are independently selected from the group consisting of —H, unsubstituted C₁-C₂₀-alkyl, and —COON; or the pair of R₉₀/R₉₁, R₉₂/R₉₃, R₉₄/R₉₅ or R₉₆/R₉₇ independently is a carbonyl

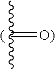

group.

In one embodiment, [symbol] is a double bond and R₉₄ and R₉₅ are independently selected from —H and —COOH.

In one embodiment, the pairs R₉₀/R₉₁ and R₉₂/R₉₃ are both carbonyl

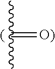

groups.

An example of a compound of formula (9) includes but is not limited to orotic acid.

### Compound of Formula (10)

In one embodiment, [symbol] is a single bond. In another embodiment, [symbol] is a double bond.

In one embodiment, V is a

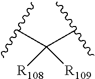

group i.e. compound (10) is a five-membered carbocyclic ring. In another embodiment, V is a —O— group. In another embodiment, V is

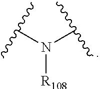

R₁₀₀, R₁₀₁, R₁₀₂, R₁₀₃, R₁₀₄, R₁₀₅, R₁₀₆, and R₁₀₇ may be independently selected from the group consisting of —H, —OH and substituted C₁-C₂₀-alkyl, wherein the substituent is one or more —OH groups.

In one embodiment, the pair of R₁₀₀/R₁₀₁, or R₁₀₆/R₁₀₇ independently is a carbonyl

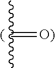

group.

In one embodiment, at least of R₁₀₀, R₁₀₁, R₁₀₂, R₁₀₃, R₁₀₄, R₁₀₅, R₁₀₆, and R₁₀₇ is a —COOH group.

Compound (10) have one or more chiral carbon atoms. Racemates, enantiomers and diastereomers are within the scope of the invention.

Examples of compounds of formula (10) include but are not limited to pyroglutamic acid (e.g. DL-, D- or L-pyroglutamic acid), camphoric acid (e.g. (+)- or (−)-camphoric acid), and ascorbic acid (e.g. DL-, D-, or L-ascorbic acid).

In one embodiment, the carboxylic acid may be selected from the group consisting of benzoic acid, fumaric acid, 3,5-dihydroxybenzoic acid, malonic acid, and oxalic acid.

In one embodiment, the carboxylic acid is not benzoic acid.

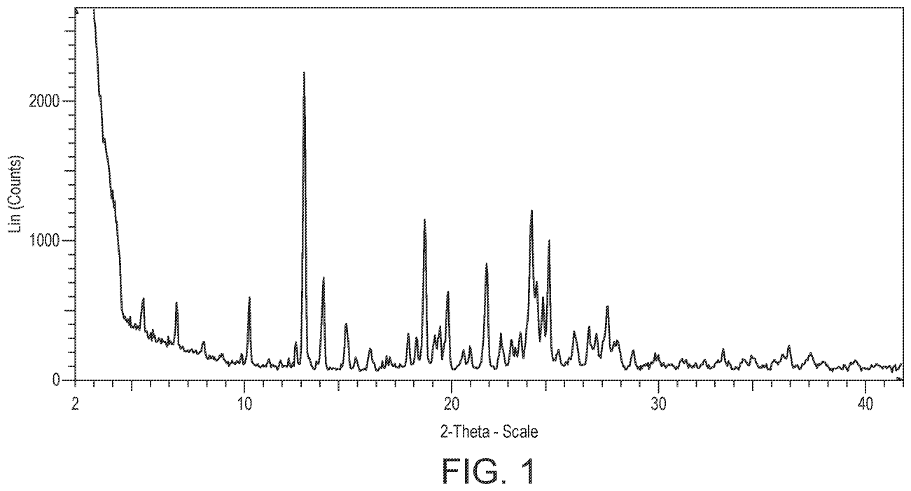

In one embodiment, the molecular complex is a crystalline metazachlor benzoic acid molecular complex. The molecular complex may have an X-ray powder diffraction pattern comprising one or more peaks (for example, 1, 2, 3, 4, 5, 6, 7, 8, 9, or 10 peaks) selected from the group consisting of about 5.2, 6.8, 8.1, 9.0, 10.0, 10.3, 11.3, 11.8, 12.3, 12.6, 13.0, 13.6, 13.9, 15.1, 15.1, 15.5, 16.2, 17.1, 18.1, 18.5, 18.9, 19.3, 19.6, 20.0, 20.7, 21.1, 21.9, 22.6, 23.1, 23.3, 23.5, 24.0, 24.3, 24.6, 24.9, 25.3, 26.1, 26.8, 27.1, 27.5, 27.7, 28.1, 29.0, 30.0, 31.3, 33.3, 34.3, 34.7, 35.8, 36.2, 36.5, and 37.6 degrees two-theta ±0.2 degrees two-theta. In one embodiment, the molecular complex may have the X-ray powder diffraction pattern substantially as shown in FIG. 1.

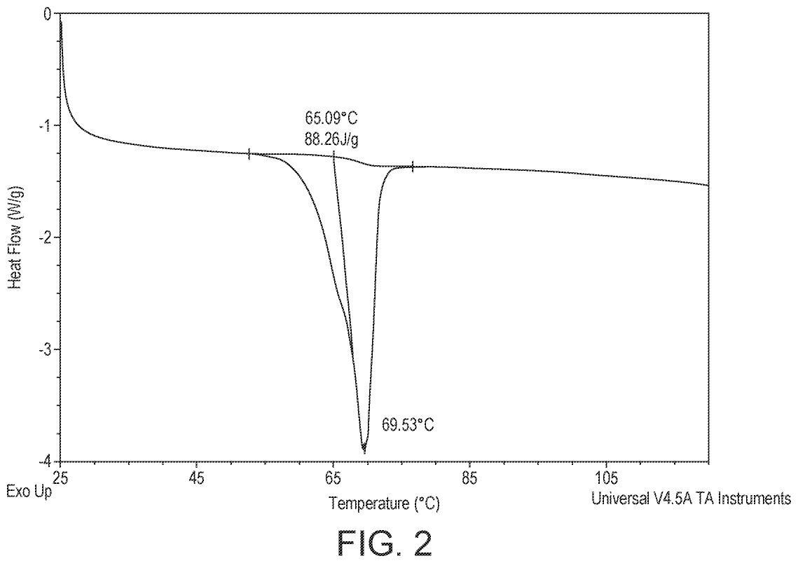

The molecular complex may have a DSC thermogram comprising an endothermal event with an onset temperature at about 65.1° C. In one embodiment, the molecular complex may have a DSC thermogram substantially as shown in FIG. 2.

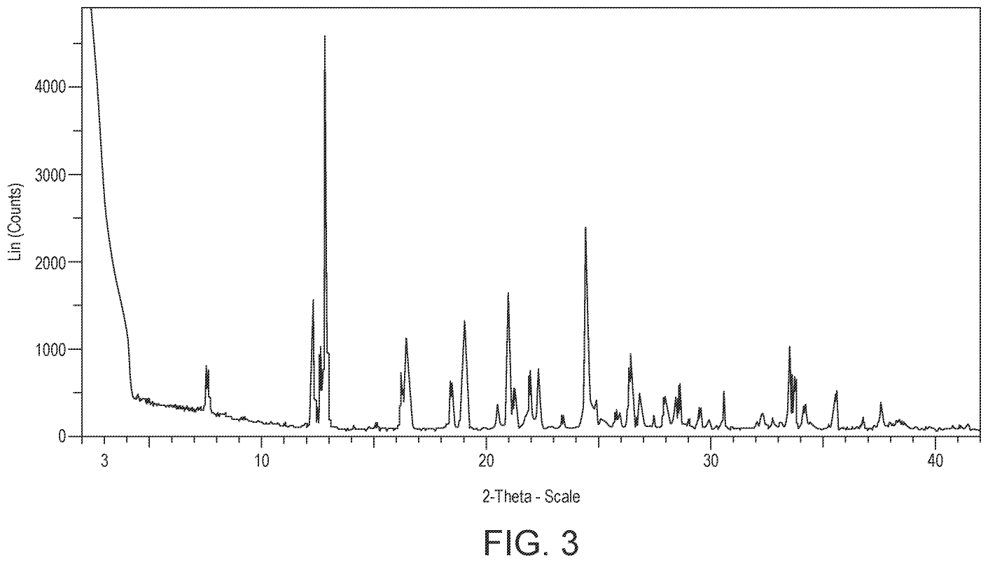

In one embodiment, the molecular complex is a crystalline metazachlor fumaric acid molecular complex. The molecular complex may have an X-ray powder diffraction pattern comprising one or more peaks (for example, 1, 2, 3, 4, 5, 6, 7, 8, 9, or 10 peaks) selected from the group consisting of about 7.5, 12.3, 12.6, 12.8, 15.1, 16.2, 16.4, 18.4, 19.0, 20.5, 21.0, 21.2, 21.8, 22.0, 22.3, 23.4, 24.4, 24.7, 24.9, 25.8, 26.0, 26.4, 26.9, 27.5, 28.0, 28.5, 28.7, 29.0, 29.5, 30.0, 30.6, 32.3, 32.8, 33.1, 33.6, 33.8, 34.2, 35.6, 36.8, and 37.6 degrees two-theta ±0.2 degrees two-theta. In one embodiment, the molecular complex may have the X-ray powder diffraction pattern substantially as shown in FIG. 3.

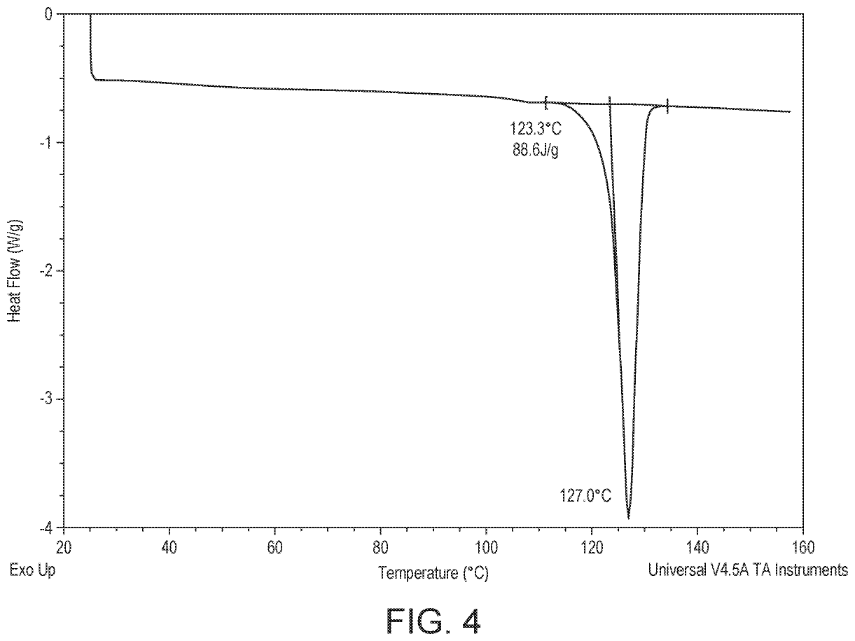

The molecular complex may have a DSC thermogram comprising an endothermal event with an onset temperature at about 123.3° C. In one embodiment, the molecular complex may have a DSC thermogram substantially as shown in FIG. 4.

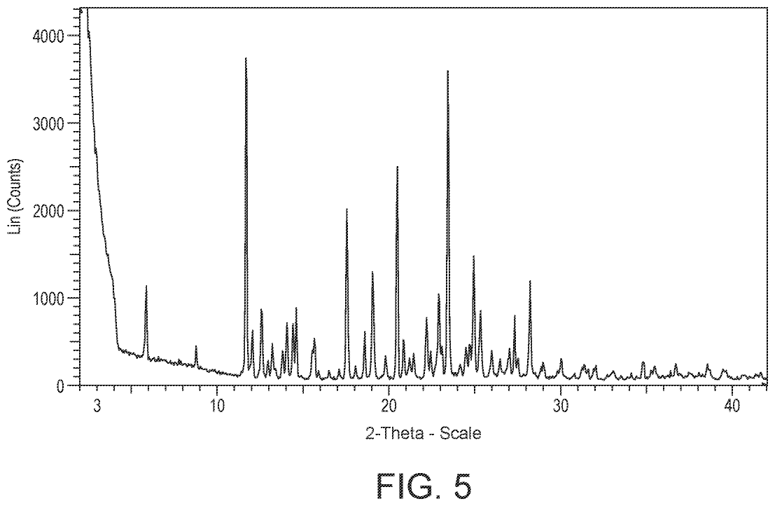

In one embodiment, the molecular complex is a crystalline metazachlor 3,5-dihydroxybenzoic acid molecular complex. The molecular complex may have an X-ray powder diffraction pattern comprising one or more peaks (for example, 1, 2, 3, 4, 5, 6, 7, 8, 9, or 10 peaks) selected from the group consisting of about 5.8, 8.8, 11.7, 12.0, 12.5, 12.9, 13.2, 13.8, 14.0, 14.4, 14.6, 15.6, 15.6, 15.9, 16.5, 17.1, 17.6, 18.1, 18.6, 19.0, 19.8, 20.5, 20.8, 21.2, 21.4, 22.2, 22.5, 22.9, 23.1, 23.5, 24.2, 24.5, 24.7, 25.0, 25.3, 26.0, 26.5, 27.0, 27.3, 27.5, 28.2, 29.0, 30.1, 31.4, 32.0, 33.1, 34.8, 35.5, 36.7, and 38.6 degrees two-theta ±0.2 degrees two-theta. In one embodiment, the molecular complex may have the X-ray powder diffraction pattern substantially as shown in FIG. 5.

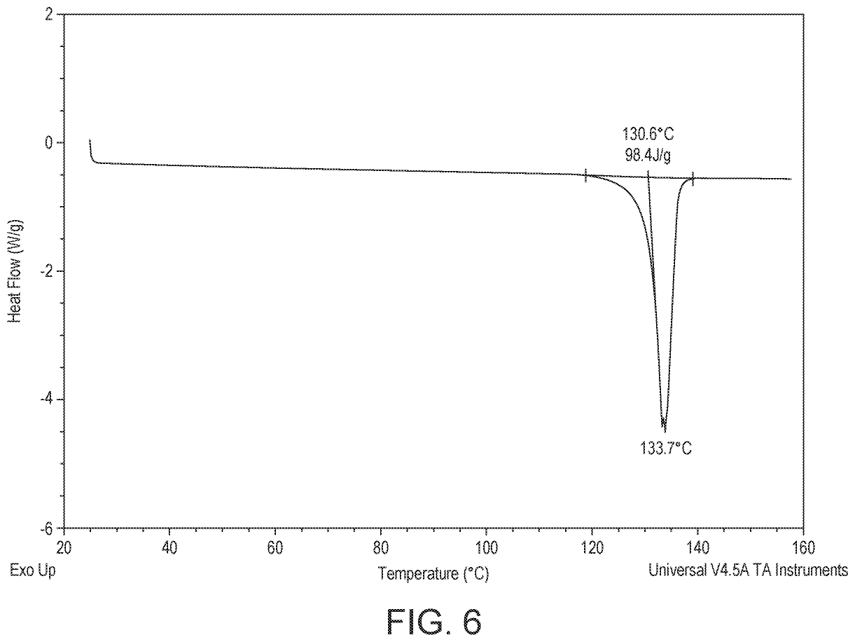

The molecular complex may have a DSC thermogram comprising an endothermal event with an onset temperature at about 130.6° C. In one embodiment, the molecular complex may have a DSC thermogram substantially as shown in FIG. 6.

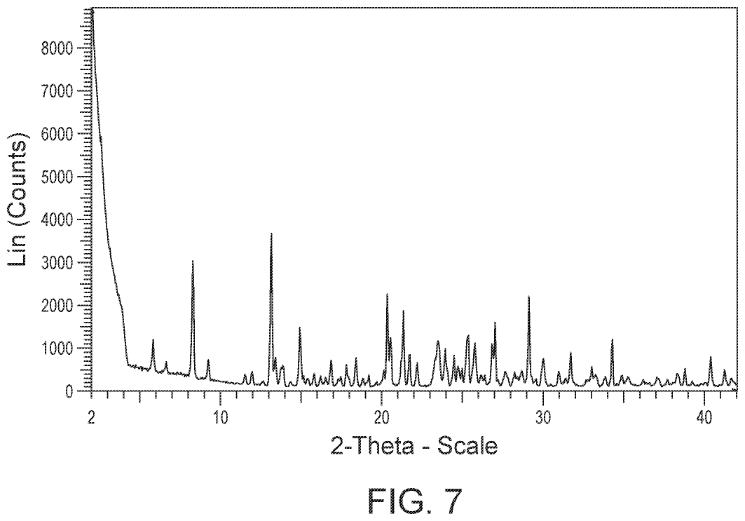

In one embodiment, the molecular complex is a crystalline metazachlor malonic acid molecular complex. The molecular complex may have an X-ray powder diffraction pattern comprising one or more peaks (for example, 1, 2, 3, 4, 5, 6, 7, 8, 9, or 10 peaks) selected from the group consisting of about 5.8, 6.6, 8.3, 9.2, 11.5, 11.9, 12.6, 13.1, 13.4, 13.8, 14.3, 14.9, 15.4, 15.8, 16.2, 16.5, 16.8, 17.4, 17.8, 18.4, 18.8, 19.2, 19.7, 20.1, 20.3, 20.5, 21.3, 21.7, 22.2, 23.5, 23.9, 24.5, 24.7, 25.0, 25.4, 25.8, 26.2, 26.4, 26.8, 27.0, 27.7, 28.3, 28.7, 29.1, 29.6, 30.0, 30.5, 31.0, 31.4, 31.7, 32.8, 33.0, 33.3, 33.8, 34.4, 34.9, 35.3, 36.2, 36.6, 37.1, 37.8, 38.3, 38.8, 39.3, 40.4, 41.3, and 41.7 degrees two-theta ±0.2 degrees two-theta. In one embodiment, the molecular complex may have the X-ray powder diffraction pattern substantially as shown in FIG. 7.

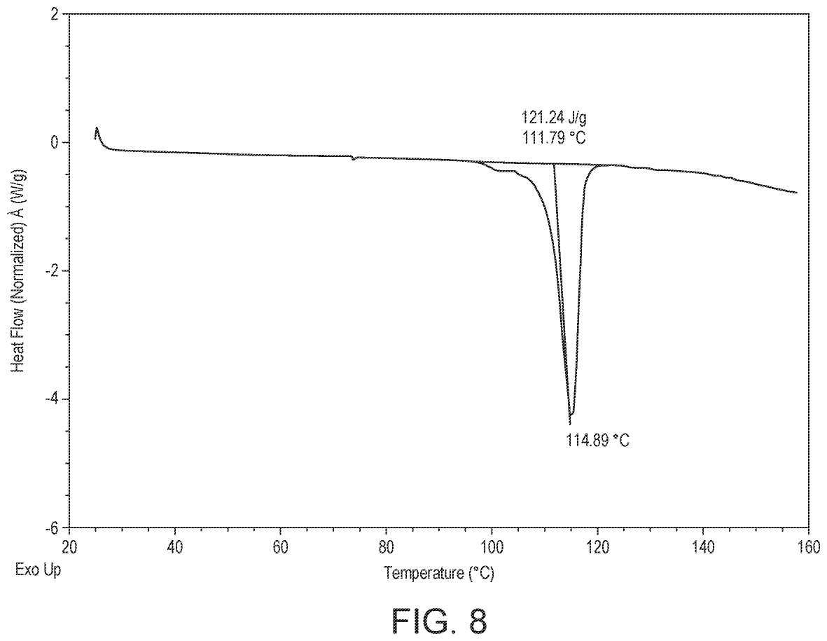

The molecular complex may have a DSC thermogram comprising an endothermal event with an onset temperature at about 121.2° C. In one embodiment, the molecular complex may have a DSC thermogram substantially as shown in FIG. 8.

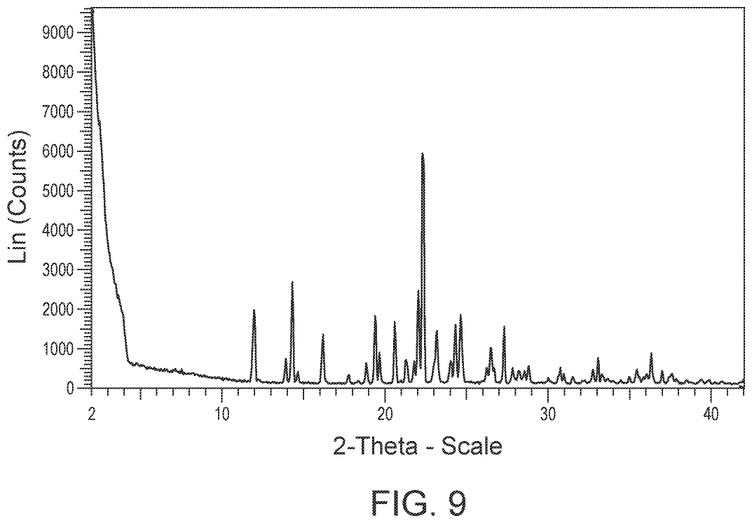

In one embodiment, the molecular complex is a crystalline metazachlor oxalic acid molecular complex. The molecular complex may have an X-ray powder diffraction pattern comprising one or more peaks (for example, 1, 2, 3, 4, 5, 6, 7, 8, 9, or 10 peaks) selected from the group consisting of about 11.9, 13.9, 14.3, 14.7, 16.2, 17.8, 18.8, 19.4, 19.7, 20.6, 21.3, 21.8, 22.0, 22.3, 23.2, 24.0, 24.3, 24.7, 26.2, 26.5, 26.7, 27.3, 27.9, 28.6, 28.8, 30.8, 31.0, 32.7, 33.1, 33.3, 35.5, 36.1, 36.4, 37.1, and 37.6 degrees two-theta ±0.2 degrees two-theta. In one embodiment, the molecular complex may have the X-ray powder diffraction pattern substantially as shown in FIG. 9.

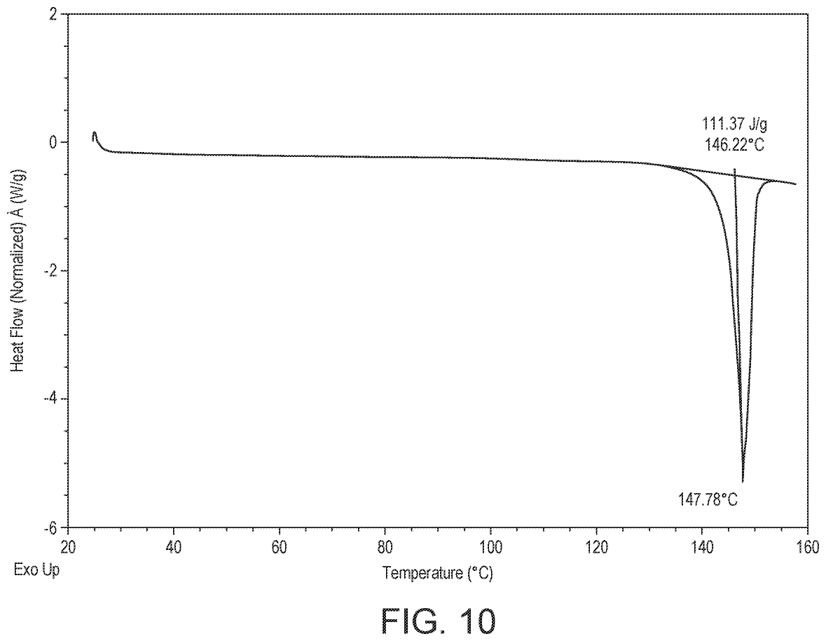

The molecular complex may have a DSC thermogram comprising an endothermal event with an onset temperature at about 146.2° C. In one embodiment, the molecular complex may have a DSC thermogram substantially as shown in FIG. 10.

The crystalline metazachlor carboxylic acid molecular complexes may be prepared by a process comprising the steps of:
(a) forming a solution of metazachlor and a carboxylic acid in a solvent selected from acetone, tetrahydrofuran (THF), or a mixture thereof;
(b) evaporating the solvent to form the crystalline molecular complex.

Metazachlor and the carboxylic acids are as described above.

In one embodiment, the solvent is acetone. In another embodiment, the solvent is THF. In yet another embodiment, the solvent is a mixture of acetone and THF.

The quantity of solvent is not particularly limiting provided there is enough solvent to substantially dissolve metazachlor and the carboxylic acid. If a suspension or hazy solution remains on contacting metazachlor and/or the carboxylic acid with the solvent, a second or further quantities of solvent may be added until a solution is formed, or the suspension or hazy solution may be filtered.

The dissolution of metazachlor and/or the carboxylic acid may be encouraged through the use of an aid such as stirring, shaking and/or sonication.

The solution of metazachlor and the carboxylic acid may formed at ambient temperature or less. Alternatively, the metazachlor and the carboxylic acid may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of about ≤35° C. to about ≤60° C. In some embodiments, the contacting step is carried out at one or more temperatures ≤about 36° C. In some embodiments, the contacting step is carried out at one or more temperatures ≤about 37° C. In some embodiments, the contacting step is carried out at one or more temperatures ≤about 38° C. In some embodiments, the contacting step is carried out at one or more temperatures ≤about 39° C. In some embodiments, the contacting step is carried out at one or more temperatures ≤about 40° C. In some embodiments, the contacting step is carried out at one or more temperatures ≤about 60° C. In some embodiments, the contacting step is carried out at one or more temperatures ≤about 59° C. In some embodiments, the contacting step is carried out at one or more temperatures ≤about 58° C. In some embodiments, the contacting step is carried out at one or more temperatures ≤about 57° C. In some embodiments, the contacting step is carried out at one or more temperatures ≤about 56° C. In some embodiments, the contacting step is carried out at one or more temperatures ≤about 55° C. In some embodiments, the contacting step is carried out at one or more temperatures ≤about 54° C. In some embodiments, the contacting step is carried out at one or more temperatures ≤about 53° C. In some embodiments, the contacting step is carried out at one or more temperatures ≤about 52° C. In some embodiments, the contacting step is carried out at one or more temperatures ≤about 51° C. In some embodiments, the contacting step is carried out at one or more temperatures ≤about 50° C. In one embodiment, the contacting step is carried out at one or more temperatures in the range of ≤about 40° C. to ≤about 50° C. In one embodiment, the contacting step is carried out at about 40° C. In another embodiment, the contacting step is carried out at about 50° C.

The crystalline molecular complex may be optionally slurried in a suitable solvent or solvent mixture in order to purify the crystalline molecular complex or remove an excess of one of the starting materials.

The solid crystalline molecular complex may be recovered directly by filtering, decanting or centrifuging.

Howsoever the crystalline molecular complex is recovered, the separated solid may be washed one or more times with a suitable solvent or solvent mixture and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the molecular complex degrades and so when the molecular complex is known to degrade within the temperature or pressure ranges given above, the drying conditions should be maintained below the degradation temperature or vacuum.

The molecular complex may be formulated into a herbicidal composition with at least one agriculturally acceptable carrier. The compositions may be solids, for example powders, granules, or water-dispersable powders, or may be liquids, such as suspensions of molecular complex particles.

Suitable agriculturally acceptable carriers are well known to those skilled in the art. Such carriers should not be phytotoxic to crops, in particular at the concentrations employed for the control of undesirable plants in the presence of crops, and should not react chemically with the compounds of the molecular complex or other composition components. The compositions may be applied directly, or may be formulations or concentrates which are diluted, for example with water, prior to application.

Liquid carriers that may be employed include water and organic solvents, although it is typically preferred that water is used. Solid carriers include mineral earths, such as clays, silicates, diatomaceous earths, or kaolin, fertilisers, and organic products such as woodmeal and cellulose carriers.

It will be understood by the skilled person that the compositions may also include further components, such as surfactants, viscosity modifiers, anti-freeze agents, agents for pH control, stabilisers and anti-caking agents.

The concentration of active ingredients in the composition is generally between about 1 and about 99 wt %, such as between about 5 and about 95 wt % or about 10 and about 90 wt %. In compositions which are designed to be diluted prior to use the concentration of active ingredients may be between about 10 and about 90 wt %. Such compositions are then diluted, for example with water, to compositions which may contain about 0.001 and about 1 wt % of active material.

The compositions as described herein may be used for controlling or substantially eliminating undesirable vegetation. Undesirable vegetation is understood to mean plants considered undesirable in a particular location, e.g. in an area of crops, and may be known as weeds.

Control may be achieved by a method comprising contacting the vegetation with the herbicidal composition. It will be understood by the skilled person that the composition at the point of application should contain a herbicidally effective amount of the molecular complex. A herbicidally effective amount is an amount of the active ingredients which causes an adverse deviation of the natural development of the undesired vegetation.

The compositions may have utility for controlling undesirable vegetation in a culture of crop plants, especially crop plants which are tolerant to the metazachlor herbicide, for example through genetic modification of the crop plants. The compositions may also have utility for the control of undesirable vegetation which is resistant to metazachlor.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

### Instrument and Methodology Details

### X-Ray Powder Diffraction (XRPD)

XRPD diffractograms were collected on a Bruker D8 diffractometer. Bruker D8 uses Cu Kα radiation (40 kV, 40 mA) and a 0-20 goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector.

The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard collection method are:
Angular range: 2 to 42° 2θ
Step size: 0.05° 2θ
Collection time: 0.5 s/step (total collection time: 6.40 min)

### DSC Method

DSC (melting point) was assessed using either a TA Instruments Q2000 or TA Instruments Discovery DSC. Typically, 0.5 -3 mg of each sample, in a pin-holed aluminium pan, was heated at 10° C./min from 25° C. to 300° C. A purge of dry nitrogen at 50 ml/min was maintained over the sample.

### Starting Materials

Metazachlor was purchased from BOC Sciences.

The carboxylic acids were purchased from Sigma Aldrich or Fluka.

### Example 1 Metazachlor: Benzoic Acid (2:1) Molecular Complex

Metazachlor (556 mg) (BOC Sciences) and benzoic acid (122 mg, 0.5 mol eq.) (Sigma-Aldrich) were dissolved in acetone (1 ml) at room temperature. The solution was left to evaporate to dryness, the resulting solid was analysed by XRPD, which showed a crystalline material and yielded a diffractogram as provided in FIG. 1.

The following table provides an XRPD peak list for the Metazachlor: benzoic acid (2:1) molecular complex:

| Angle | Intensity |
|---|---|
| (2-Theta °) | (%) |
| 5.2 | 26.3 |
| 6.8 | 25.0 |
| 8.1 | 12.1 |
| 9.0 | 8.2 |
| 10.0 | 8.2 |
| 10.3 | 26.6 |
| 11.3 | 6.5 |
| 11.8 | 6.1 |
| 12.3 | 6.8 |
| 12.6 | 12.0 |
| 13.0 | 100.0 |
| 13.6 | 6.3 |
| 13.9 | 33.1 |
| 15.1 | 13.4 |
| 15.1 | 18.1 |
| 15.5 | 7.0 |
| 16.2 | 9.9 |
| 17.1 | 7.2 |
| 18.1 | 14.9 |
| 18.5 | 13.6 |
| 18.9 | 52.1 |
| 19.3 | 14.3 |
| 19.6 | 17.2 |
| 20.0 | 28.4 |
| 20.7 | 9.4 |
| 21.1 | 10.6 |
| 21.9 | 37.7 |
| 22.6 | 14.9 |
| 23.1 | 12.8 |
| 23.3 | 9.8 |
| 23.5 | 15.2 |
| 24.0 | 54.9 |
| 24.3 | 31.8 |
| 24.6 | 26.6 |
| 24.9 | 45.3 |
| 25.3 | 9.6 |
| 26.1 | 15.5 |
| 26.8 | 17.2 |
| 27.1 | 14.7 |
| 27.5 | 13.9 |
| 27.7 | 23.7 |
| 28.1 | 12.1 |
| 29.0 | 9.3 |
| 30.0 | 8.4 |
| 31.3 | 6.6 |
| 33.3 | 9.8 |
| 34.3 | 6.5 |
| 34.7 | 7.7 |
| 35.8 | 6.2 |
| 36.2 | 8.1 |
| 36.5 | 10.8 |
| 37.6 | 8.5 |

The molecular complex was also characterised by DSC (FIG. 2). DSC analysis indicated a melting point with an onset temperature of about 65.1° C.

The molecular complex was further analysed by single crystal x-ray crystallography to determine the crystal structure as follows:

### Data collection and Structure Refinement For Metazachlor: Benzoic Acid Molecular Complex

| | |
|---|---|
| Diffractometer | SuperNova, Dual, Cu at zero, Atlas |
| Radiation source | SuperNova (Cu) X-ray Source, CuKα |
| Data collection method | omega scans |
| Theta range for data | 3.472 to 74.528° |
| collection | |
| Index ranges | −9 ≤ h ≤ 9, −15 ≤ k ≤ 16, −21≤/≤21 |
| Reflections collected | 32210 |
| Independent reflections | 6827 [R(int) = 0.0341] |
| Coverage of independent | 100.0% |
| reflections | |
| Variation in check | n/a |
| reflections | |
| Absorption correction | Semi-empirical from equivalents |
| Max. and min. transmission | 1.00000 and 0.76217 |
| Structure solution technique | Direct methods |
| Structure solution program | SHELXL-2013 (Sheldrick, 2013) |
| Refinement technique | Full-matrix least-squares on F² |
| Refinement program | SHELXL-2013 (Sheldrick, 2013) |
| Function minimized | Σw(Fₒ² − F_{c}²)² |
| Data/restraints/parameters | 6827/0/432 |
| Goodness-of-fit on F² | 1.046 |
| Δ/σₘₐₓ | 0.001 |
| Final R indices | |
| 6170 data; I > 2σ(I) | R1 = 0.0366, wR2 = 0.0982 |
| all data | R1 = 0.0408, wR2 = 0.1020 |
| Weighting scheme | w = 1/[σ² (F_{c}²) + (0.0580 P)² + 0.6500 P] |
| | where P = (Fₒ² − 2F_{c}²)²/3 |
| Extinction coefficient | n/a |
| Largest diff. peak and hole | 0.431 and −0.323 eÅ⁻³ |

### Sample and Crystal Data For Metazachlor: Benzoic Acid Molecular Complex

| Compound | Metazachlor-benzoic acid molecular complex_(2:1) | |
|---|---|---|
| Crystallization | Acetone | |
| solvents | | |
| Crystallization | Evaporation | |
| method | | |
| Empirical formula | C₃₅H₃₈Cl₂N₆O₄ | |
| Formula weight | 677.61 | |
| Temperature | 100(2) K | |
| Wavelength | 1.54184 Å | |
| Crystal size | 0.420 × 0.180 × 0.040 mm | |
| Crystal habit | colourless shard | |
| Crystal system | Triclinic | |
| Space group | P1 | |
| Unit cell dimensions | a = 7.7461(2) Å | α = 96.229(2)° |
| | b = 12.8182(3) Å | β = 91.892(2)° |
| | c = 17.1130(4) Å | γ = 92.269(2)° |

| | |
|---|---|
| Volume | 1686.62(7) Å³ |
| Z | 2 |
| Density (calculated) | 1.334 Mg/m³ |
| Absorption | 2.123 mm⁻¹ |
| coefficient | |
| F(000) | 712 |

Example 2 Metazachlor: Fumaric Acid (2:1) Molecular Complex M

etazachlor (556 mg) (BOC Sciences) and fumaric acid (116 mg, 0.5 mol eq.) (Fluka) were stirred at 50° C. in THF (12 ml) at room temperature, a light suspension was obtained and filtered. The solution was left to evaporate to dryness at room temperature. The resulting solid was analysed by XRPD, which showed a crystalline material and yielded a diffractogram as provided in FIG. 3.

The following table provides an XRPD peak list for the Metazachlor: fumaric acid (2:1) molecular complex:

| Angle | Intensity |
|---|---|
| (2-Theta °) | (%) |
| 7.5 | 17.5 |
| 12.3 | 34.2 |
| 12.6 | 22.3 |
| 12.8 | 100.0 |
| 15.1 | 3.2 |
| 16.2 | 15.4 |
| 16.4 | 24.0 |
| 18.4 | 13.7 |
| 19.0 | 29.0 |
| 20.5 | 7.6 |
| 21.0 | 36.4 |
| 21.2 | 12.0 |
| 21.8 | 6.9 |
| 22.0 | 16.5 |
| 22.3 | 16.9 |
| 23.4 | 5.5 |
| 24.4 | 52.5 |
| 24.7 | 13.3 |
| 24.9 | 9.0 |
| 25.8 | 6.7 |
| 26.0 | 5.8 |
| 26.4 | 20.3 |
| 26.9 | 10.6 |
| 27.5 | 5.1 |
| 28.0 | 10.0 |
| 28.5 | 9.8 |
| 28.7 | 13.1 |
| 29.0 | 4.5 |
| 29.5 | 7.2 |
| 30.0 | 4.0 |
| 30.6 | 11.2 |
| 32.3 | 5.9 |
| 32.8 | 4.7 |
| 33.1 | 3.5 |
| 33.6 | 22.2 |
| 33.8 | 15.1 |
| 34.2 | 8.2 |
| 35.6 | 11.7 |
| 36.8 | 4.9 |
| 37.6 | 8.7 |

The molecular complex was also characterised by DSC (FIG. 4). DSC analysis indicated a melting point with an onset temperature of about 123.3° C.

The sample was further analysed by single crystal x-ray crystallography to determine the crystal structure as follows:

### Data collection and Structure Refinement For Metazachlor: Fumaric Acid Molecular Complex

| | |
|---|---|
| Diffractometer | SuperNova, Dual, Cu at zero, Atlas |
| Radiation source | SuperNova (Cu) X-ray Source, CuKα |
| Data collection method | omega scans |
| Theta range for data | 3.775 to 74.510° |
| collection | |
| Index ranges | −17 ≤ h ≤ 17, −10 ≤ k ≤ 9, −17≤/≤18 |
| Reflections collected | 16261 |
| Independent reflections | 3355 [R(int) = 0.0441] |
| Coverage of independent | 99.9% |
| reflections | |
| Variation in check | n/a |
| reflections | |
| Absorption correction | Semi-empirical from equivalents |
| Max. and min. transmission | 1.00000 and 0.84504 |
| Structure solution technique | Direct methods |
| Structure solution program | SHELXTL (Sheldrick, 2013) |
| Refinement technique | Full-matrix least-squares on F² |
| Refinement program | SHELXL-2013 (Sheldrick, 2013) |
| Function minimized | Σw(Fₒ² − F_{c}²)² |
| Data/restraints/parameters | 3355/0/214 |
| Goodness-of-fit on F² | 1.044 |
| Δ/σₘₐₓ | 0.001 |
| Final R indices | |
| 6170 data; I > 2σ(I) | R1 = 0.0402, wR2 = 0.1056 |
| all data | R1 = 0.0460, wR2 = 0.1125 |
| Weighting scheme | w = 1/[σ² (Fₒ²) + (0.0632 P)² + 0.8464 P] |
| | where P = (Fₒ² − 2F_{c}²)²/3 |
| Extinction coefficient | n/a |
| Extinction coefficient | n/a |
| Largest diff. peak and hole | 0.615 and −0.716 eÅ⁻³ |

### Sample and Crystal Data For Metazachlor-Fumaric Acid Molecular Complex

| Compound | Metazachlor-fumaric acid molecular complex (2:1) | |
|---|---|---|
| Crystallization | Acetone | |
| solvents | | |
| Crystallization | Evaporation | |
| method | | |
| Empirical formula | C₁₆H₁₈ClN₃O₃ | |
| Formula weight | 335.78 | |
| Temperature | 100(2) K | |
| Wavelength | 1.54184 Å | |
| Crystal size | 0.800 × 0.300 × 0.130 mm | |
| Crystal habit | Colourless irregular block | |
| Crystal system | Monoclinic | |
| Space group | P2₁/n | |
| Unit cell dimensions | a = 14.3457(4) Å | α = 90° |
| | b = 8.31831 (16) Å | β = 107.938(3)° |
| | c = 14.6209(3) Å | γ = 90° |

| | |
|---|---|
| Volume | 1659.93(7) Å³ |
| Z | 4 |
| Density (calculated) | 1.344 Mg/m³ |
| Absorption | 2.198 mm⁻¹ |
| coefficient | |
| F(000) | 704 |

### Example 3 Metazachlor: 3,5-dihydroxybenzoic Acid (2:1) Molecular Complex

Metazachlor (556 mg) (BOC Sciences) and 3,5-dihydroxybenzoic acid (154 mg, 0.5 mol eq.) (Sigma Aldrich) were stirred in THF (3 ml) at 40° C. for 30 min. The resulting hazy solution was filtered and left to evaporate. The resulting solid was analysed by XRPD, which showed a crystalline material and yielded a diffractogram as provided in FIG. 5.

The following table provides an XRPD peak list for the Metazachlor: 3,5 dihydroxybenzoic acid (2:1) molecular complex:

| Angle | Intensity |
|---|---|
| (2-Theta °) | (%) |
| 5.8 | 30.2 |
| 8.8 | 11.7 |
| 11.7 | 100.0 |
| 12.0 | 17.8 |
| 12.5 | 24.2 |
| 12.9 | 7.3 |
| 13.2 | 12.4 |
| 13.8 | 10.3 |
| 14.0 | 18.7 |
| 14.4 | 18.5 |
| 14.6 | 23.3 |
| 15.6 | 11.0 |
| 15.6 | 14.1 |
| 15.9 | 4.2 |
| 16.5 | 4.2 |
| 17.1 | 4.6 |
| 17.6 | 53.4 |
| 18.1 | 5.6 |
| 18.6 | 16.0 |
| 19.0 | 34.4 |
| 19.8 | 8.8 |
| 20.5 | 66.0 |
| 20.8 | 13.9 |
| 21.2 | 8.1 |
| 21.4 | 9.4 |
| 22.2 | 20.3 |
| 22.5 | 10.2 |
| 22.9 | 27.5 |
| 23.1 | 12.0 |
| 23.5 | 95.6 |
| 24.2 | 6.1 |
| 24.5 | 11.3 |
| 24.7 | 12.3 |
| 25.0 | 39.2 |
| 25.3 | 22.6 |
| 26.0 | 10.2 |
| 26.5 | 7.7 |
| 27.0 | 11.0 |
| 27.3 | 20.9 |
| 27.5 | 8.1 |
| 28.2 | 31.5 |
| 29.0 | 6.8 |
| 30.1 | 7.8 |
| 31.4 | 6.1 |
| 32.0 | 5.8 |
| 33.1 | 4.2 |
| 34.8 | 6.9 |
| 35.5 | 5.6 |
| 36.7 | 6.3 |
| 38.6 | 6.3 |

The molecular complex was also characterised by DSC (FIG. 6). DSC analysis indicated a melting point with an onset temperature of about 130.6° C.

### Example 4 Metazachlor: Malonic Acid (2:1) Molecular Complex

Metazachlor (200 mg) (BOC Sciences) and malonic acid (38 mg, 0.5 mol eq.) (Sigma-Aldrich) were dissolved in THF (1 ml) at room temperature. The clear solution was left to evaporate to dryness. The resulting solid was analysed by XRPD, which showed a crystalline material and yielded a diffractogram as provided in FIG. 7.

The following table provides an XRPD peak list for the Metazachlor: malonic acid (2:1) molecular complex:

| Angle | Intensity |
|---|---|
| (2-Theta °) | (%) |
| 5.8 | 32.5 |
| 6.6 | 18.1 |
| 8.3 | 82.5 |
| 9.2 | 19.9 |
| 11.5 | 10.0 |
| 11.9 | 12.0 |
| 12.6 | 5.7 |
| 13.1 | 100.0 |
| 13.4 | 21.1 |
| 13.8 | 15.7 |
| 14.3 | 5.1 |
| 14.9 | 40.4 |
| 15.4 | 7.5 |
| 15.8 | 11.3 |
| 16.2 | 9.4 |
| 16.5 | 8.7 |
| 16.8 | 19.3 |
| 17.4 | 8.5 |
| 17.8 | 16.6 |
| 18.4 | 20.8 |
| 18.8 | 7.6 |
| 19.2 | 10.1 |
| 19.7 | 5.4 |
| 20.1 | 13.0 |
| 20.3 | 61.3 |
| 20.5 | 33.7 |
| 21.3 | 51.1 |
| 21.7 | 22.8 |
| 22.2 | 17.3 |
| 23.5 | 31.5 |
| 23.9 | 26.3 |
| 24.5 | 22.2 |
| 24.7 | 15.1 |
| 25.0 | 14.1 |
| 25.4 | 34.8 |
| 25.8 | 30.3 |
| 26.2 | 9.7 |
| 26.4 | 9.7 |
| 26.8 | 29.5 |
| 27.0 | 43.9 |
| 27.7 | 11.7 |
| 28.3 | 11.6 |
| 28.7 | 12.4 |
| 29.1 | 59.9 |
| 29.6 | 7.1 |
| 30.0 | 20.1 |
| 30.5 | 3.9 |
| 31.0 | 12.4 |
| 31.4 | 7.5 |
| 31.7 | 24.0 |
| 32.8 | 6.5 |
| 33.0 | 14.8 |
| 33.3 | 10.2 |
| 33.8 | 8.6 |
| 34.4 | 32.4 |
| 34.9 | 9.4 |
| 35.3 | 8.5 |
| 36.2 | 7.0 |
| 36.6 | 4.8 |
| 37.1 | 8.6 |
| 37.8 | 6.9 |
| 38.3 | 10.7 |
| 38.8 | 13.8 |
| 39.3 | 5.7 |
| 40.4 | 21.2 |
| 41.3 | 13.4 |
| 41.7 | 7.5 |

The molecular complex was also characterised by DSC (FIG. 8). DSC analysis indicated a melting point with an onset temperature of about 121.2° C.

### Example 5 Metazachlor: Oxalic Acid (2:1) Molecular Complex

Metazachlor (200 mg) (BOC Sciences) and oxalic acid (32 mg, 0.5 mol eq.) (Sigma-Aldrich) was dissolved in THF (1 ml) at room temperature. The clear solution was left to evaporate to dryness. The resulting solid was analysed by XRPD, which showed a crystalline solid and yielded the diffractogram as provided in FIG. 9.

The following table provides an XRPD peak list for the Metazachlor: oxalic acid (2:1) molecular complex:

| Angle | Intensity |
|---|---|
| (2-Theta °) | (%) |
| 11.9 | 32.8 |
| 13.9 | 12.6 |
| 14.3 | 44.8 |
| 14.7 | 7.2 |
| 16.2 | 22.5 |
| 17.8 | 5.2 |
| 18.8 | 10.8 |
| 19.4 | 30.5 |
| 19.7 | 14.6 |
| 20.6 | 28.4 |
| 21.3 | 12.0 |
| 21.8 | 11.5 |
| 22.0 | 41.5 |
| 22.3 | 100.0 |
| 23.2 | 24.1 |
| 24.0 | 11.5 |
| 24.3 | 27.1 |
| 24.7 | 31.6 |
| 26.2 | 8.9 |
| 26.5 | 16.9 |
| 26.7 | 8.6 |
| 27.3 | 25.9 |
| 27.9 | 8.2 |
| 28.6 | 7.6 |
| 28.8 | 9.2 |
| 30.8 | 8.3 |
| 31.0 | 6.0 |
| 32.7 | 7.4 |
| 33.1 | 12.4 |
| 33.3 | 5.9 |
| 35.5 | 7.7 |
| 36.1 | 6.2 |
| 36.4 | 14.6 |
| 37.1 | 6.9 |
| 37.6 | 6.5 |

The molecular complex was also characterised by DSC (FIG. 10 Error! Reference source not found.). DSC analysis indicated a melting point with an onset temperature of about 146.2° C.

1. A crystalline molecular complex comprising metazachlor and a carboxylic acid.

2. A crystalline molecular complex according to claim 1, wherein the carboxylic acid is selected from the group consisting of:
(a) the compound of formula (2): 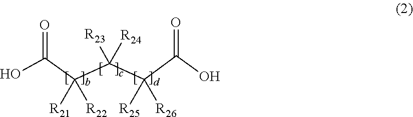
wherein:
b is an integer which is 0, 1, 2, 3, 4, or 5;
c is an integer which is 0, 1, 2, 3, 4, or 5;
d is an integer which is 0, 1, 2, 3, 4, or 5;
R₂₁, R₂₂, R₂₃, R₂₄, R₂₅, and R₂₆ are independently selected from the group consisting of —H, —OH, —NH₂ and —COOH; or
the pair of R₂₁/R₂₂, R₂₃/R₂₄ and/or R₂₅/R₂₆ independently is a carbonyl 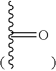 [SMILES: *=O]
group.
(b) the compound of formula (3): 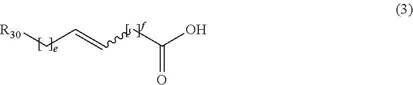
wherein:
e is an integer selected from 0, 1, 2, 3, 4, 5, 6, 7, or 8;
f is an integer selected from 0, 1, 2, 3, 4, 5, 6, 7, or 8;
the symbol [symbol] denotes that the stereochemistry of the C═C double bond is cis- or trans-;
R₃₀ is selected from the group consisting of —H, —CO₂H, unsubstituted C₁-C₂₀-alkyl, substituted C₁-C₂₀-alkyl, unsubstituted C₅-C₂₀-aryl, and substituted C₅-C₂₀-aryl.
(c) the compound of formula (4): 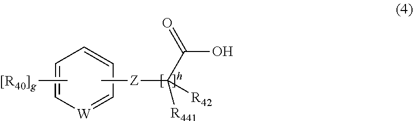
wherein:
g is an integer selected from 0, 1, 2, 3, 4 or 5;
h is an integer selected from 0, 1, 2, 3, or 4;
W is a carbon atom or a nitrogen atom;
Z is absent or is a —CO—NH— group;
R₄₀ is selected from the group consisting of —OH, —NH₂, and —NH—CO—R₄₃;
R₄₁ and R₄₂ are independently selected from the group consisting of —H, —OH, unsubstituted C₁-C₂₀-alkyl, substituted C₁-C₂₀-alkyl, unsubstituted C₅-C₂₀-aryl, and substituted C₅-C₂₀-aryl;
R₄₃ is selected from the group consisting of unsubstituted C₁-C₂₀-alkyl, substituted C₁-C₂₀-alkyl, unsubstituted C₅-C₂₀-aryl, and substituted C₅-C₂₀-aryl;
provided that when W is a nitrogen atom, g is an integer selected from 0, 1, 2, 3, or 4.
(d) the compound of formula (5): 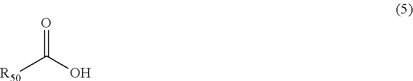
wherein:
R₅₀ is selected from the group consisting of —H, unsubstituted C₁-C₂₀-alkyl, and substituted C₁-C₂₀-alkyl.
(e) the compound of formula (6): 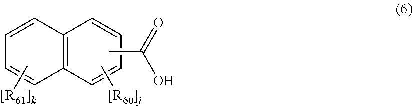
wherein:
j is an integer selected from 0, 1, 2 or 3;
k is an integer selected from 0, 1, 2, 3, or 4;
R₆₀ and R₆₁ are independently selected from the group consisting of —H, unsubstituted C₁-C₂₀-alkyl, substituted C₁-C₂₀-alkyl, unsubstituted C₅-C₂₀-aryl, substituted C₅-C₂₀-aryl, unsubstituted —(C₁-C₂₀-alkyl)-(C₅-C₂₀-aryl), and substituted —(C₁-C₂₀-alkyl)-(C₅-C₂₀-aryl).
(f) the compound of formula (7): 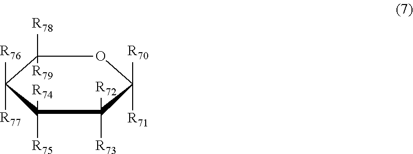
wherein:
R₇₉, R₇₁, R₇₂, R₇₃, R₇₄, R₇₅, R₇₆, R₇₇, R₇₈ and R₇₉ are independently selected from the group consisting of —H, —OH and —COOH, provided that at least one of R₇₀, R₇₁, R₇₂, R₇₃, R₇₄, R₇₅, R₇₆, R₇₇, R₇₈ and R₇₉ is —COOH.
(g) the compound of formula (8): 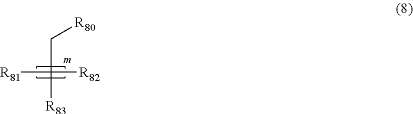
wherein:
m is an integer selected from 1, 2, 3, 4, 5, 6, 7, 8, 9, or 10;
R₈₀, R₈₁, R₈₂ and R₈₃ are independently selected from —H, —OH, —COOH and R₈₄;
R₈₄ is the group: 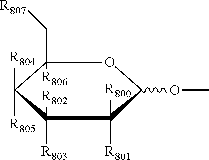
wherein:
R₈₀₀, R₈₀₁, R₈₀₂, R₈₀₃, R₈₀₄, R₈₀₅, R₈₀₆ and R₈₀₇ are independently selected from the group consisting of —H, —OH, and —COOH;
provided that at least one of R₈₀, R₈₁, R₈₂, R₈₃, R₈₀₀, R₈₀₁, R₈₀₂, R₈₀₃, R₈₀₄, R₈₀₅, R₈₀₆ and R₈₀₇ is —COOH.
(h) the compound of formula (9): 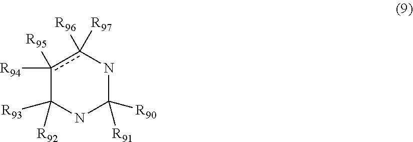
wherein: [symbol]
is a single or double bond;
R₉₀, R₉₁, R₉₂, R₉₃, R₉₄, R₉₅, R₉₆, and R₉₇ are independently selected from the group consisting of —H, unsubstituted C₁-C₂₀-alkyl, substituted C₁-C₂₀-alkyl, and —COOH; or
the pair of R₉₀/R₉₁, R₉₂/R₉₃, R₉₄/R₉₅ and/or R₉₆/R₉₇ independently is a carbonyl 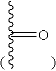 [SMILES: *=O]
group;
provided that:
(i) when [symbol] is a double bond, R₉₅ and R₉₇ are absent;
(ii) when the pair of R₉₄/R₉₅ and/or R₉₆/R₉₇ is a carbonyl 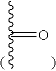 [SMILES: *=O]
group, [symbol] is a single bond;
(iii) at least one of R₉₀, R₉₁, R₉₂, R₉₃, R₉₄, R₉₅, R₉₆, and R₉₇ is —COOH.
(g) the compound of formula (10): 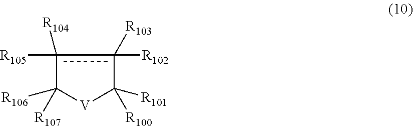
wherein: [symbol]
is a single or double bond;
V is —O—, 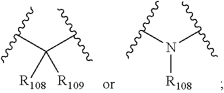
R₁₀₀, R₁₀₁, R₁₀₂, R₁₀₃, R₁₀₄, R₁₀₅, R₁₀₆, R₁₀₇, R₁₀₈, R₁₀₉ and R₁₁₀ are independently selected from the groups consisting of —H, —OH, unsubstituted C₁-C₂₀-alkyl, substituted C₁-C₂₀-alkyl, and —COOH; or
the pair of R₁₀₀/R₁₀₁, R₁₀₂/R₁₀₃, R₁₀₄/R₁₀₅ or R₁₀₆/R₁₀₇ independently is a carbonyl 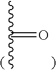 [SMILES: *=O]
group;
provided that:
(i) when [symbol] is a double bond, R₁₀₂ and R₁₀₅ are absent; and
(ii) when the pair of R₁₀₄/R₁₀₅ and/or R₁₀₆/R₁₀₇ is a carbonyl 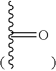 [SMILES: *=O]
group, [symbol] is a single bond.

3. A crystalline molecular complex according to claim 2, wherein the carboxylic acid is selected from the group consisting of benzoic acid, fumaric acid, 3,5-dihydroxybenzoic acid, malonic acid, and oxalic acid.

4. A crystalline molecular complex according to claim 3, wherein the molecular complex is a crystalline metazachlor benzoic acid molecular complex having an X-ray powder diffraction pattern comprising one or more peaks selected from the group consisting of about 5.2, 6.8, 8.1, 9.0, 10.0, 10.3, 11.3, 11.8, 12.3, 12.6, 13.0, 13.6, 13.9, 15.1, 15.1, 15.5, 16.2, 17.1, 18.1, 18.5, 18.9, 19.3, 19.6, 20.0, 20.7, 21.1, 21.9, 22.6, 23.1, 23.3, 23.5, 24.0, 24.3, 24.6, 24.9, 25.3, 26.1, 26.8, 27.1, 27.5, 27.7, 28.1, 29.0, 30.0, 31.3, 33.3, 34.3, 34.7, 35.8, 36.2, 36.5, and 37.6 degrees two-theta ±0.2 degrees two-theta.

5. A crystalline molecular complex according to claim 4, which has the X-ray powder diffraction pattern substantially as shown in FIG. 1.

6. A crystalline molecular complex according to claim 3, wherein the molecular complex is a crystalline metazachlor fumaric acid molecular complex having an X-ray powder diffraction pattern comprising one or more peaks selected from the group consisting of about 7.5, 12.3, 12.6, 12.8, 15.1, 16.2, 16.4, 18.4, 19.0, 20.5, 21.0, 21.2, 21.8, 22.0, 22.3, 23.4, 24.4, 24.7, 24.9, 25.8, 26.0, 26.4, 26.9, 27.5, 28.0, 28.5, 28.7, 29.0, 29.5, 30.0, 30.6, 32.3, 32.8, 33.1, 33.6, 33.8, 34.2, 35.6, 36.8, and 37.6 degrees two-theta ±0.2 degrees two-theta.

7. A crystalline molecular complex according to claim 6, which has the X-ray powder diffraction pattern substantially as shown in FIG. 3.

8. A crystalline molecular complex according to claim 3, the molecular complex is a crystalline metazachlor 3,5-dihydroxybenzoic acid molecular complex having an X-ray powder diffraction pattern comprising one or more peaks selected from the group consisting of about 5.8, 8.8, 11.7, 12.0, 12.5, 12.9, 13.2, 13.8, 14.0, 14.4, 14.6, 15.6, 15.6, 15.9, 16.5, 17.1, 17.6, 18.1, 18.6, 19.0, 19.8, 20.5, 20.8, 21.2, 21.4, 22.2, 22.5, 22.9, 23.1, 23.5, 24.2, 24.5, 24.7, 25.0, 25.3, 26.0, 26.5, 27.0, 27.3, 27.5, 28.2, 29.0, 30.1, 31.4, 32.0, 33.1, 34.8, 35.5, 36.7, and 38.6 degrees two-theta ±0.2 degrees two-theta.

9. A crystalline molecular complex according to claim 8, which has the X-ray powder diffraction pattern substantially as shown in FIG. 5.

10. A crystalline molecular complex according to claim 3, wherein the molecular complex is a crystalline metazachlor malonic acid molecular complex having an X-ray powder diffraction pattern comprising one or more peaks selected from the group consisting of about 5.8, 6.6, 8.3, 9.2, 11.5, 11.9, 12.6, 13.1, 13.4, 13.8, 14.3, 14.9, 15.4, 15.8, 16.2, 16.5, 16.8, 17.4, 17.8, 18.4, 18.8, 19.2, 19.7, 20.1, 20.3, 20.5, 21.3, 21.7, 22.2, 23.5, 23.9, 24.5, 24.7, 25.0, 25.4, 25.8, 26.2, 26.4, 26.8, 27.0, 27.7, 28.3, 28.7, 29.1, 29.6, 30.0, 30.5, 31.0, 31.4, 31.7, 32.8, 33.0, 33.3, 33.8, 34.4, 34.9, 35.3, 36.2, 36.6, 37.1, 37.8, 38.3, 38.8, 39.3, 40.4, 41.3, and 41.7 degrees two-theta ±0.2 degrees two-theta.

11. A crystalline molecular complex according to claim 10, which has the X-ray powder diffraction pattern substantially as shown in FIG. 7.

12. A crystalline molecular complex according to claim 3, wherein the molecular complex is a crystalline metazachlor oxalic acid molecular complex having an X- ray powder diffraction pattern comprising one or more peaks selected from the group consisting of about 11.9, 13.9, 14.3, 14.7, 16.2, 17.8, 18.8, 19.4, 19.7, 20.6, 21.3, 21.8, 22.0, 22.3, 23.2,

24. 0, 24.3, 24.7, 26.2, 26.5, 26.7, 27.3, 27.9, 28.6, 28.8, 30.8, 31.0, 32.7, 33.1, 33.3, 35.5, 36.1, 36.4, 37.1, and 37.6 degrees two-theta ±0.2 degrees two-theta.

13. A crystalline molecular complex according to claim 12, which has the X-ray powder diffraction pattern substantially as shown in FIG. 9.

14. A process for preparing a crystalline molecular complex according to claim 1, the process comprising the steps of:
(a) forming a solution of metazachlor and a carboxylic acid in a solvent selected from acetone, tetrahydrofuran (THF), or a mixture thereof;
(b) evaporating the solvent to form the crystalline molecular complex.

15. A herbicidal composition comprising a molecular complex as claimed in claim 1 and at least one agriculturally acceptable carrier.

16. A herbicidal composition according to claim 15, wherein the composition is an aqueous suspension or granules.

17. The use of an herbicidal composition according to claim 15 for controlling or substantially eliminating undesired vegetation.

18. A method for controlling undesired vegetation comprising contacting the vegetation with a herbicidal composition according to claim 15.
